(19) United States
(12) Patent Application Publication
(10) Pub. No.: US 2023/0035969 A1
(43) Pub. Date: Feb. 2, 2023

(54) DEVICES, SYSTEMS, AND METHODS FOR STABILIZING MEDICAL TUBING PROTRUDING FROM A PATIENT

(71) Applicant: HAITEC Medical, Inc., Rancho Santa Fe, CA (US)

(72) Inventors: Adam M. ARIELY, Encintas, CA (US); Juraj LETKO, Chicago, IL (US)

(21) Appl. No.: 17/877,301

(22) Filed: Jul. 29, 2022

Related U.S. Application Data
(60) Provisional application No. 63/227,855, filed on Jul. 30, 2021.

(51) Int. Cl. A61M 25/02

(52) U.S. Cl. CPC A61M 25/02; A61M 2025/0206; A61M 2025/0273

(57) ABSTRACT

Devices, systems, and methods for stabilizing medical tubing protruding from a patient are disclosed herein. In various embodiments of the present disclosure, a medical tubing stabilization device is provided that may include a tube stabilization patch and a complementary medical garment. The tube stabilization patch may be applied to a patient who has medical tubing or a catheter protruding from their body to reduce or dampen movement on the body from the medical tubing in relation to the patient. The medical garment may be worn by the patient and placed in combination with the tube stabilization patch to further reduce the relative movement of the medical tubing. The present disclosure may be directed to reducing the level of medical tubing movement, both externally and internally, that a patient may experience, which decreases the risk of infection caused by catheterization of patients.

### RELATED APPLICATION

This application claims the benefit of U.S. Provisional Application No. 63/227,855 filed on Jul. 30, 2021 the contents of which are incorporated herein by reference in their entirety.

### TECHNICAL FIELD

The present disclosure relates generally to devices, systems, and methods for stabilizing medical tubing protruding from or into a patient's body. In particular, some implementations may relate to such devices, systems, and methods that prevent or reduce relative movement between the medical tubing and the patient by dampening forces acting on the body caused by the movement and/or pull of the tubing.

### BACKGROUND

Many areas of medicine involve fluid drainage from a patient's tissues, organs, or cavities. Usually this involves the use of flexible medical tubing extending from tissues, organs, and/or cavities, which are connected to a fluid collection device outside of or fastened to the patient. Conventional devices, systems, and methods are problematic as they allow for lateral, longitudinal, and/or internal movement of the tubing or fluid collection device in relation to the patient insertion or attachment site. Such lateral, longitudinal, and/or internal movement can cause trauma, contamination, and infection, both at the tubing's insertion sites (e.g., skin, cavity, organ, genitalia, urethra, etc.) as well as in associated tissues. Conventional systems that allow for such movement may introduce contaminating microorganisms from the exterior surface of the tubing into the patient's tissues and vasculature due to a portion of the tubing adjacent to the patient moving in to and out of (i.e., “pistons”) the patient.

The problems associated with the movement of catheters, particularly urinary catheters such as in-dwelling (or Foley catheters), suprapubic urinary catheters, ileostomy catheters, chest tubes, and the like, are particularly pronounced, because the medical tubing used to drain the affected organ or tissue protrude directly outward from the patient. These tubes tend to be of larger diameter, have heavier wall thickness, and tend to be more rigid than venous catheter tubes. Although more rigid than venous catheter tubes, typical urinary catheter tubes are still flexible medical tubing, usually made from Latex, a Latex blend, and/or silicone, and pass through the urethra into the bladder.

In-dwelling urinary catheters are typically retained inside the bladder by a balloon located at the inserted end of the catheter and are often inflated with sterile water or saline solution. The diameter of the inflated balloon is much greater than the internal diameter of the urethra, which can assist in holding the catheter inside the patient's body. Catheter diameters are typically sized by the French (Fr) catheter scale, which is a measure of a catheter's outer diameter, with the French size being three times the outer diameter, measured in millimeters (mm) (e.g., 3 Fr). Typical urinary catheters range from 10 Fr (3.3 mm) to 28 Fr (9.3 mm). The external end of the catheter is typically attached to a drainage tube that leads to a drainage (or collection) bag. The catheter and/or drainage tube may be conventionally secured to one of the patient's legs to help control catheter and drainage tubing movement and attempt to mitigate the resultant effects of the movement of the catheter on the patient. To minimize tension on the catheter, care must be taken when securing the drainage tube to ensure that a sufficient length of slack tubing is available to form a curve or loop in the tubing. If insufficient tubing slack is available, the risk of bladder damage increases. Slack drainage tubing, however, creates a problem where, while the inflated catheter balloon reduces the risk of the catheter from accidentally being pulled out of the bladder, nothing restricts the catheter from moving further into the bladder. Because of the extra length that slack tubing provides, the catheter (and balloon) can move within the bladder every time a patient's leg moves. Additionally, catheters have a natural rigidity (i.e., it wants to be straight), thus the extra slack in the catheter does not stay curved and results in the tip of the catheter pushing into the lining of the bladder because the opposing end of the catheter (the portion external to the body) is fixed by the leg securement device. Should the catheter move far enough into the bladder, the top and/or internal end (e.g., tip that extends beyond the balloon in the bladder) of the catheter contacts the bladder lining opposite the urethra, which may cause bladder spasms as well as other trauma. Repeated movement of the catheter at the opening of the urethra can also cause urethral discomfort, irritation, and/or trauma, in addition to increasing a patient's risk of contracting a catheter-associated urinary tract infection (CAUTI).

According to the Centers for Disease Control and Prevention (CDC), each year in the U.S., 30 million urinary catheters are inserted into over 5 million patients in acute care hospitals and extended care facilities. Up to 25% of these patients, over one million patients per year, develop CAUTI when subject to catheterization for at least 7 days, with the daily risk amounting to 5%. CAUTI is the most common nosocomial infection, and the second most common cause of nosocomial blood stream infection. Studies suggest that patients who develop CAUTI have an increased institutional death rate, unrelated, but in addition to, an increased risk of developing urosepsis. As the population grows in size and increases in age, and as average life expectancy increases, each year more people will require catheterization. These factors, in addition to the increasing unwillingness of the U.S. Centers for Medicare and Medicaid (alongside a growing number of health insurance companies) to pay hospitals for nosocomial infection treatment, creates an urgent need for additional solutions for the management and stabilization of medical tubing, including in-dwelling urinary catheters, suprapubic urinary catheters, ileostomy catheters, chest tubes, and the like.

### SUMMARY

Devices, systems, and methods are described herein for a medical tubing stabilization device. The medical tubing stabilization device may include a tube stabilization patch, which further may include a tube engaging element that can be configured to receive medical tubing; a patient attachment element that may be coupled to the tube engaging element; and an access separation, which may allow for medical tubing to pass from outside the tube stabilization patch, through the patient attachment element, and into the tube engaging element.

The medical tubing stabilization device may further include a medical garment. Such medical garment may have a body that is wrapped around the patient. The body may include an adjustable flap that is secured to a first portion of the body and an attachment mechanism that allows for the adjustable strap to be releasably connected to a second portion of the body. The adjustable flap may also include a securing mechanism that may both hold the tube stabilization patch and the tube engaging element in place. The securing mechanism may have an opening or exit port that may allow for the tube engaging element to be secured between portions of the securing mechanism.

Other features and aspects of the disclosure will become apparent from the following detailed description, taken in conjunction with the accompanying figures, which illustrate, by way of example, the features in accordance with various embodiments. The summary is not intended to limit the scope of the invention, which is defined solely by the claims attached hereto.

### BRIEF DESCRIPTION OF THE FIGURES

The technology disclosed herein, in accordance with one or more various embodiments, is described in detail with reference to the following figures. The figures are provided for purposes of illustration only and merely depict typical or example embodiments of the disclosed technology. These figures are provided to facilitate the reader's understanding of the disclosed technology and shall not be considered limiting of the breadth, scope, or applicability thereof. It should be noted that for clarity and ease of illustration, these figures are not necessarily made to scale.

FIG. 1 is a front view of an example medical garment of a device or system for stabilizing medical tubing protruding from a patient with an adjustable flap in an unattached position, according to embodiments of the present disclosure.

FIG. 2 is a front view of an example medical garment of a device or system for stabilizing medical tubing protruding from a patient with an adjustable flap in an attached position, according to embodiments of the present disclosure.

FIG. 3 is a front view of an example medical garment of a device or system for stabilizing medical tubing protruding from a patient with an adjustable flap in an unattached position and a catheter securing mechanism in an unsecured position with a closure flap open, according to embodiments of the present disclosure.

FIG. 4 is a front view of an example medical garment of a device or system for stabilizing medical tubing protruding from a patient with an adjustable flap in a partially attached position and a catheter securing mechanism in an unsecured position with a closure flap open, according to embodiments of the present disclosure.

FIG. 5 is a front view of an example medical garment of a device or system for stabilizing medical tubing protruding from a patient with an adjustable flap in an attached position and a catheter securing mechanism in a secured position with a closure flap closed, according to embodiments of the present disclosure.

FIG. 6 is a perspective view of an example tube stabilization patch of a device or system for stabilizing medical tubing protruding from a patient, according to embodiments of the present disclosure.

FIGS. 7a & 7b are example views of patients with suprapubic or ileostomy catheters secured by a tube stabilization patch of a device or system for stabilizing medical tubing protruding from a patient, according to embodiments of the present disclosure.

FIG. 8 is a perspective view of an example medical garment of a device or system for stabilizing medical tubing protruding from a patient with an adjustable flap in an unattached position and a catheter securing mechanism in a secured position with a closure flap closed, with example medical tubing protruding from a patient, according to embodiments of the present disclosure.

FIG. 9 is a perspective view of an example medical garment of a device or system for stabilizing medical tubing protruding from a patient with an adjustable flap in an unattached position and a catheter securing mechanism in an unsecured position with a closure flap open, wherein example medical tubing protruding from a patient is passed between a first portion and a second portion of the catheter securing mechanism, according to embodiments of the present disclosure.

FIG. 10 is a perspective view of an example medical garment of a device or system for stabilizing medical tubing protruding from a patient with an adjustable flap in a partially attached position and a catheter securing mechanism in an unsecured position with a closure flap open, wherein example medical tubing protruding from a patient is passed between a first portion and a second portion of the catheter securing mechanism and situated at a catheter exit port, according to embodiments of the present disclosure.

FIG. 11 is a perspective view of an example medical garment of a device or system for stabilizing medical tubing protruding from a patient with an adjustable flap in a partially attached position and a catheter securing mechanism in an unsecured position with a closure flap open, wherein example medical tubing protruding from a patient is passed through the tube stabilization patch and disposed between the body of the patient and the first and second portion of the catheter securing mechanism, according to embodiments of the present disclosure.

FIG. 12 is a perspective view of an example medical garment of a device or system for stabilizing medical tubing protruding from a patient with an adjustable flap in an attached position and a catheter securing mechanism in a secured position with a closure flap open, wherein example medical tubing protruding from a patient is passed through the tube stabilization patch and disposed between the body of the patient and the first and second portion of the catheter securing mechanism, according to embodiments of the present disclosure.

FIG. 13 is a perspective view of an example medical garment of a device or system for stabilizing medical tubing protruding from a patient with an adjustable flap in an attached position and a catheter securing mechanism in a secured position with a closure flap closed, wherein example medical tubing protruding from a patient is passed through the tube stabilization patch and disposed between the body of the patient and the first and second portion of the catheter securing mechanism, according to embodiments of the present disclosure.

FIG. 14 is a perspective view of an example medical garment of a device or system for stabilizing medical tubing protruding from a patient with an adjustable flap in an attached position and a catheter securing mechanism in a secured position with a closure flap closed, wherein example medical tubing protruding from a patient is passed through the first and second portion of the catheter securing mechanism and then passed through the tube stabilization patch with the tube stabilization patch adhered to an external portion of the medical garment, according to embodiments of the present disclosure.

FIG. 15 is a rear view of an example medical garment of a device or system for stabilizing medical tubing protruding from a patient with a posterior attachment member, according to embodiments of the present disclosure.

FIG. 16 is a side view of an example medical garment of a device or system for stabilizing medical tubing protruding from a patient with an adjustable flap attached to the posterior attachment member, according to embodiments of the present disclosure.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

The present disclosure relates to devices, systems, and methods for management and stabilization of medical tubing. In particular, various embodiments may relate to management and stabilization for in-dwelling urinary catheters, suprapubic urinary catheters, ileostomy catheters, chest tubes, and the like.

As discussed, the problem with protruding medical tubing and catheters is that they are free to move laterally around the patient insertion site or entry point on the patient; move in and out (piston) of the patient, which introduces microbials and bacteria; and further move around the entry point, which may cause callusing, strain, or other tissue damage.

The present disclosure provides a solution via a medical tubing stabilization device. The provided medical tubing stabilization device may include a medical garment and a tube stabilization patch. The tube stabilization patch may include a tube engaging element that holds the tube in place and a patient attachment element connected to the periphery or perimeter of the tube engaging element, which attaches the tube stabilization patch to the patient.

The tube stabilization patch reduces the amount of relative movement of the medical tubing or catheter by providing an additional structural component around the medical tubing or catheter. Such a structure (i.e., the tube engaging element) may be more rigid than conventional medical tubing or catheters in order to reduce the amount of free lateral movement the catheter can engage in. Additionally, the tube engaging element may secure the medical tubing or catheter, such that the medical tubing or catheter movement in and out (i.e., “piston”) of the patient is significantly reduced. By reducing the piston effect, less bacteria and microbials are introduced into the interior of the patient's body, resulting in fewer instances of CAUTI. Additionally, due to the decreased lateral movement, the patient may experience reduced levels of strain on the entry point of the medical tubing or catheter. As a result, reduced piston effect and reduced lateral movement, reduce the amount of medical tubing or catheter movement internal to the patient, which prevents tissue damage and other irritation. The tube stabilization patch may be made from single-use or disposable materials, and may be provided to patients in a sterile and/or sealed container to further reduce the risk of possible exposure to bacteria and microbials.

The patient attachment element may be configured for removable attachment to a region of skin on a patient or to an external surface of the medical garment (as discussed below). Since the patient attachment element is connected to the periphery of the tube engaging element, a patient can easily remove the tube stabilization patch by pulling on the patient attachment element. In some embodiments, a pull tab may be included on the patient attachment element to further ease the patient's removal of the tube stabilization patch.

In embodiments, once the medical tubing, or for example, an in-dwelling urinary catheter, is positioned in the patient, the tube engaging element may be attached to the tubing or catheter by, for example, snapping it on to or clamping it over, the medical tubing at a location at or near where the tubing exits the patient's body. When the tube engaging element of the tube stabilization patch is releasably attached to the medical tubing or catheter, the tube stabilization patch can control, i.e., prevent or reduce, the relative movement of the medical tubing in relation to the stabilization device.

The tube stabilization patch may be further secured with a medical garment or undergarment. The medical garment may include a body portion that wraps around a patient and an adjustable flap that secures to an anterior section of the body portion. The adjustable flap may include a securing mechanism that may allow for the medical tubing or catheter to pass through such securing mechanism without having to be removed from the patient. The securing mechanism may be secured around the tube engaging element, further increasing the stabilization of the catheter. By combining the stabilization effect of the tube stabilization patch and the medical garment, the lateral movement and piston effect experienced by patients may be decreased.

Additionally, the medical garment may be secured onto the patient and the tube stabilization patch may then be secured around the medical tubing and secured to an external surface of the medical garment. This may provide a similar dampening affect as attached the tube stabilization patch to the patient or beneath the medical garment, but may allow for the patch to be more easily changed. In other words, the tube stabilization patch may be disposed between the body of the patient and the medical garment (i.e., body-patch-garment) or the medical garment may be disposed between the body of the patient and the tube stabilization patch (i.e., body-garment-patch).

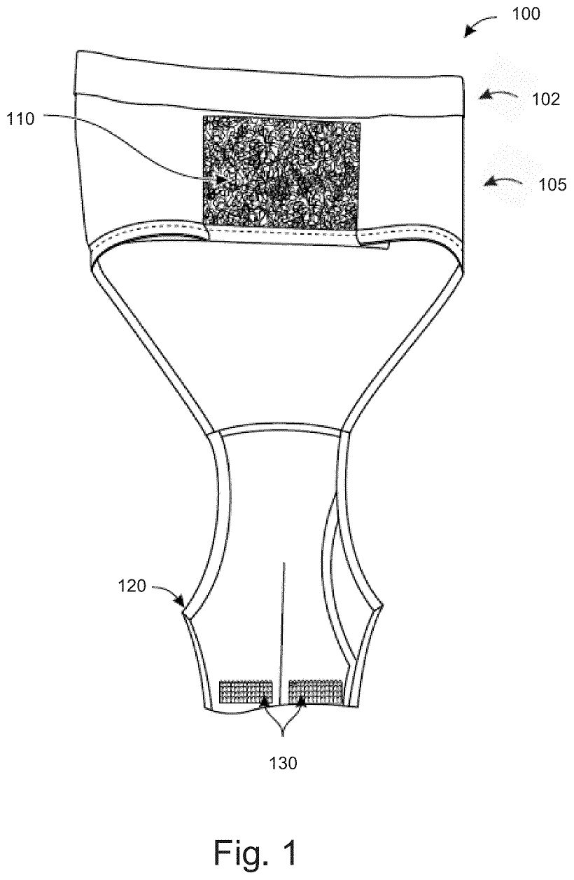

Now turning to the Figures. FIG. 1 is a front view of an example medical garment of a device or system for stabilizing medical tubing protruding from a patient with an adjustable flap in an unattached position, according to embodiments of the present disclosure.

According to various embodiments of the present disclosure, the medical garment 100 may include an encircling waistband 102 and an encircling body portion 105. The encircling body portion 105 may include a rear portion configured to cover a dorsal area below the waistband 102 along with a patient's buttocks region. The body portion 105 may further extend to cover a lower portion that connects to a front portion comprising an adjustable flap 120. The body portion 105 and front portion may be connected by non-partable or partable seams, or may further be connected by structure. For example, the body portion 105 and front portion may be cut from a singular piece of fabric and thus connected by the structure of the fabric.

The front portion comprising an adjustable flap 120 may extend from the lower region of the rear portion beneath the crotch area of the patient to a front portion of the medical garment 100. The portion comprising an adjustable flap 120 may have a right side and a left side that define, in combination with the encircling body portion 105, a right leg opening and a left leg opening, respectively.

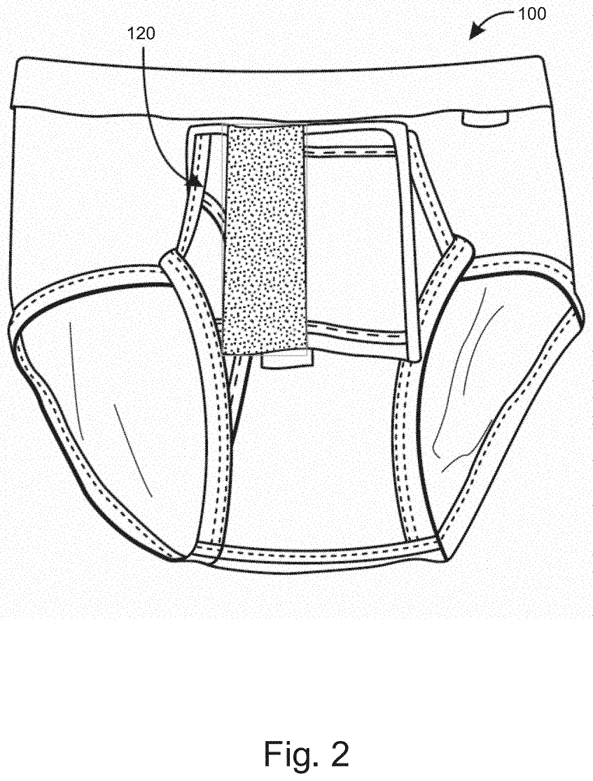

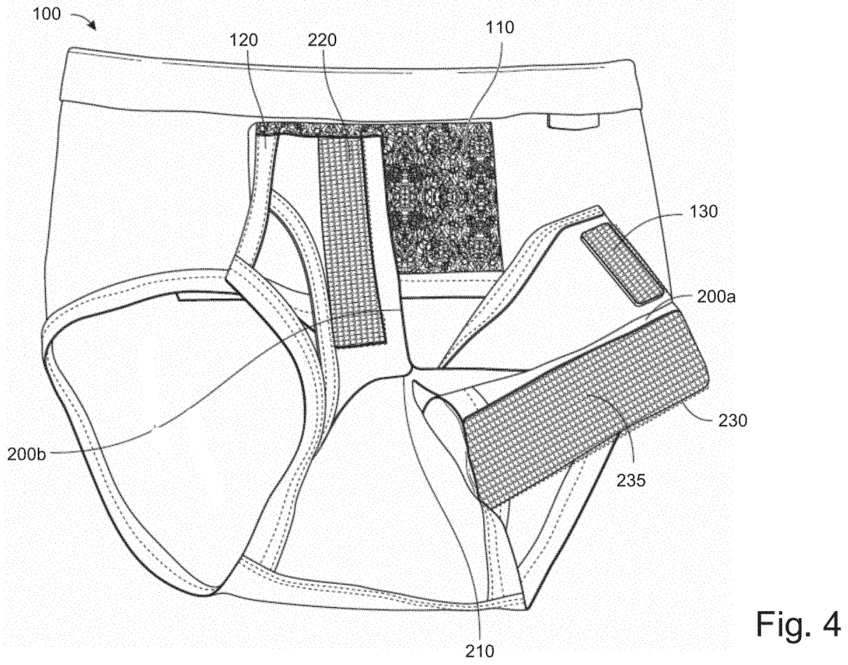

The front portion comprising an adjustable flap 120 may be configured to cover an anterior area of the patient's abdomen below the waistband 102. The section of the front portion comprising an adjustable flap that is free from connection to the body portion (i.e., the section or end of the front portion opposite the section or end of the front portion connected to the rear portion) may include an attachment mechanism. The adjustable flap 120 may be unattached from the body portion 105, as depicted in FIG. 1, attached, as depicted in FIG. 2, or partially attached as depicted in FIG. 4.

To attach the adjustable flap 120 to the body portion 105, an attachment mechanism is provided. The attachment mechanism may include a first attachment portion 110 and a second attachment portion 130. The first attachment portion 110 may be disposed on the anterior area of the body portion 105 below the waistband 102. The first attachment portion 110 may have a height and a width that allows for the adjustable flap 120 to be attached a various positions along the first attachment portion 110, which may account for varying patient anatomy. In various embodiment, the adjustable flap affects the location of the medical tubing exit and having increased area for securing the second attachment portion 130 to the first attachment portion 110 allows for increased levels of comfort and a neutral position of medical tubing when worn by patients. For example, the first attachment portion 110 may be 4 inches tall and 5 inches wide, which may allow for different sized patients to wear the medical garment and adjust the adjustable flap for the greatest level of comfort.

The second attachment portion 130 may be disposed on the side of the adjustable flap 120 that, when attached (discussed in detail below), faces internally towards the patient. The first attachment portion 110 and the second attachment portion 130 may, for example, attach to each other via a conventional hook and loop method, Velcro, reusable adhesive, and/or any other suitable method for repeatably attaching fabrics to one another. As discussed, the first attachment portion 110 may have a width and height. In some embodiments, the height may exceed the width. In other embodiments, the width may exceed the height. In further embodiments, the width and height may be the same. The second attachment portion 130 may have a width and height. In some embodiments, the height may exceed the width. In other embodiments, the width may exceed the height. In further embodiments, the width and height may be the same.

Referring to FIG. 2, this figure depicts a front view of an example medical garment of a device or system for stabilizing medical tubing protruding from a patient with an adjustable flap in an attached position, according to embodiments of the present disclosure.

According to various embodiments of the present disclosure, the medical garment 100 may have an adjustable flap 120. As described in relation to FIG. 1, the adjustable flap 120 may be in an unattached position (see 120 in FIG. 1, where the adjustable flap 120 is unattached from the body portion 105). When the adjustable flap 120 is unattached, it may allow easier access for a patient to don the medical garment, as it can be slid over a catheter without disrupting the catheter position by maneuvering legs of the patient into fixed leg holes, as would be required by conventional garments. Once the body of the medical garment is around the patient's abdomen, the adjustable flap 120 can be attached, via the attachment mechanism, to the body of the medical garment 100.

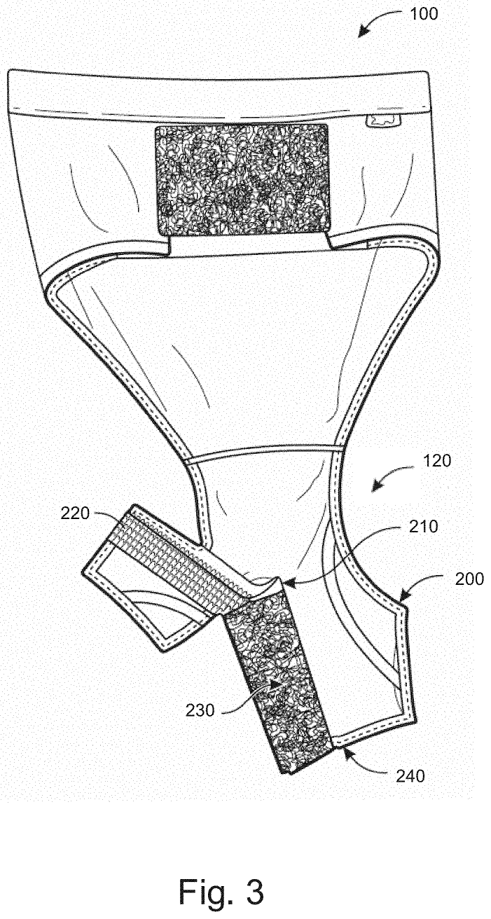

Referring to FIG. 3, this figure depicts a front view of an example medical garment of a device or system for stabilizing medical tubing protruding from a patient with an adjustable flap in an unattached position and a catheter securing mechanism in an unsecured position with a closure flap open, according to embodiments of the present disclosure.

According to various embodiments of the present disclosure, the adjustable flap 120 on the medical garment 100 may include a securing mechanism 200 (i.e., a catheter securing mechanism). The securing mechanism 200 may include a catheter exit port 210, which in embodiments may be an opening, slit, tunnel, or otherwise unincumbered area that allows for a medical tube or catheter to pass through said opening. The securing mechanism 200 may also have a first securing portion 220 and a second securing portion 230, which may connect or secure together via conventional hook and loop methods, Velcro, adhesive, or other known methods of repeatably securing fabric together. The first securing portion 220 may have a width and height. In some embodiments, the height may exceed the width. In other embodiments, the width may exceed the height. In further embodiments, the width and height may be the same. The second securing portion 230 may have a width and height. In some embodiments, the height may exceed the width. In other embodiments, the width may exceed the height. In further embodiments, the width and height may be the same.

The example depicted in FIG. 3 shows a medical garment 100 with the adjustable flap 120 unattached and the securing mechanism 200 unsecured because the first securing portion 220 and the second securing portion 230 are not attached, connected, or secured to each other aside from at a common base point near or about the catheter exit port 210. The securing mechanism 200 may allow for a catheter to pass through the catheter exit port 210 when the first securing portion 220 and the second securing portion 230 are secured, unsecured, or partially secured (as described in greater detail below).

The securing mechanism 200 may also include various securing mechanism surfaces 240. The securing mechanism surfaces may define a structure of the securing mechanism 200, which are not defined by the first securing portion 220, the second securing portion 230, or the catheter exit port 210. When the adjustable flap 120 is attached to the body of the medical garment 100, the securing mechanism surface 240 may be disposed on the anterior pubis region of the patient, which may provide a stabilization effect on the patient's abdominal region, pubis region, or protruding medical tubing (e.g., urinary catheter).

Referring to FIG. 4, this figure depicts a front view of an example medical garment of a device or system for stabilizing medical tubing protruding from a patient with an adjustable flap in a partially attached position and a catheter securing mechanism in an unsecured position with a closure flap open, according to embodiments of the present disclosure.

According to various embodiments of the present disclosure, the medical garment 100, as depicted in FIG. 4, may include an adjustable flap 120 in a partially attached position, where only a part of the second attachment portion 130 is attached to the first attachment portion 110. The partially attached position may occur when the securing mechanism 200 is unsecured, i.e., the first securing portion 220 on a first side of the securing mechanism 200a is attached via the attachment mechanism 120 to the body of the medical garment 100, and the second securing portion 230 on a second side of the securing mechanism 200b is both unsecured from the first securing portion 220 and unattached from the body of the medical garment 100.

The first securing portion 220 and the second securing portion 230 on a surface 235 may have a conventional hook and loop element, Velcro, adhesive, or any other repeatable means for securing fabric together. The conventional hook and loop element may cover the entirety or a portion of the surface of the first securing portion 220 and the second securing portion 230.

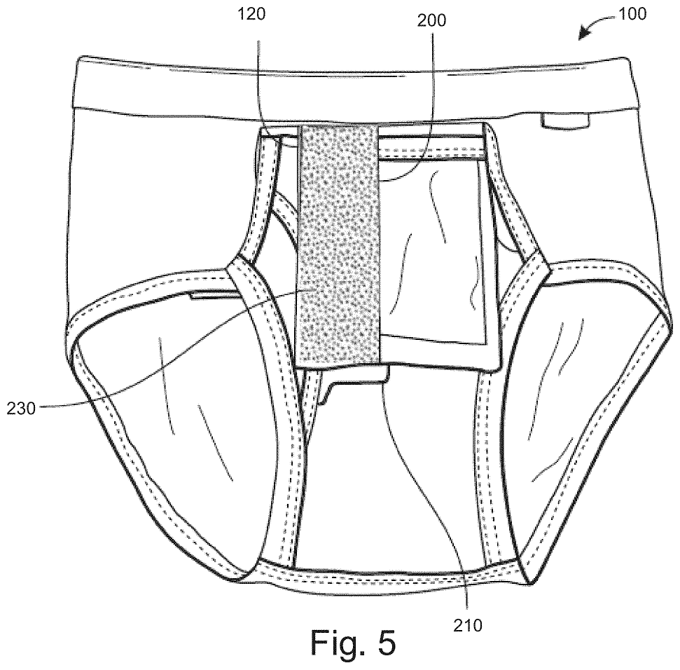

The example depicted in FIG. 4, may occur when a patient is donning the medical garment and begins to secure the securing mechanism 200 around medical tubing (as discussed below), before the medical garment 100 is fully secured on the patient (as depicted in FIG. 5).

Referring to FIG. 5, this figure depicts a front view of an example medical garment of a device or system for stabilizing medical tubing protruding from a patient with an adjustable flap in an attached position and a catheter securing mechanism in a secured position with a closure flap closed, according to embodiments of the present disclosure.

According to various embodiments of the present disclosure, the medical garment 100 may be fully secured on a patient when the adjustable flap 120 is attached to the body of the medical garment 100, the first securing portion 220 is secured to the second securing portion 230 on the securing mechanism 200, and the catheter exit port 210 is situated at or about the desired location for protruding medical tubing or a catheter to exit the medical garment.

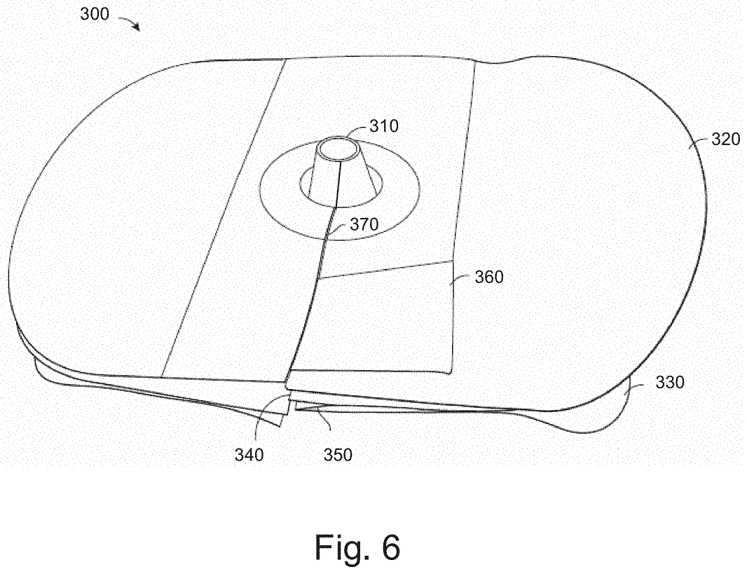

FIG. 6 is a perspective view of an example tube stabilization patch of a device or system for stabilizing medical tubing protruding from a patient, according to embodiments of the present disclosure.

According to various embodiments of the present disclosure, a tube stabilization patch 300 may be provided. The tube stabilization patch 300 may include a tube engaging element 310, a patient attachment element 320, and an access separation 370.

The tube engaging element 310 may include an openable bore that may be configured to engage an external surface of medical tubing or a urinary catheter. The openable bore may extend through the tube engaging element 310 and prevent or reduce relative movement of the medical tubing or urinary catheter in relation to the patient, the tube engaging element 310, the tube stabilization patch 300, or similar. The tube stabilization patch may reduce the amount of relative movement (lateral, longitudinal, “pistoning”) of the medical tubing or catheter when the medical tubing or catheter is situated in the openable bore of the tube engaging element 310.

The openable bore may be configured to engage an external surface of a medical tube (e.g., a catheter). In some embodiments, the openable bore may be configured to engage an external surface of a tubular medical port or a retaining element releasably and securely attached to the medical drainage tube in order to prevent or reduce relative movement of the medical tubing in relation to the tube or tube stabilization patch 300. This stabilization may occur when the medical tubing is disposed or positioned in the tube engaging element's 310 openable tunnel. In embodiments, the medical tubing may be placed or positioned through the openable bore from back to front or front to back, perpendicular to the orientation of the tube stabilization patch 300.

In some embodiments, the tube engaging element 310 may be comprised of two or more openable bore segments that, when joined (e.g., by clamping, snapping, etc.) form the openable bore.

In some embodiments, the tube engaging element 310 may be a resilient material formed into an openable sidewall. The resilient material may be rubber (natural, synthetic, or a combination thereof), silicone, any suitable plastic, or a combination thereof. The resilience of the tube engaging element 310 allows for the tube stabilization patch 300 to dampen movement on the body cause by movement of the medical tubing or catheter when one or more of longitudinal, lateral, or piston forces are applied to the tubing or the catheter. Additionally, the tube engaging element 310 may be coated with an antibacterial or antimicrobial agent, which can further reduce the risk of infection in patients.

The patient attachment element 320 may be connected to a perimeter of the tube engaging element 310. Any suitable set of complementary structural or mechanical features can be used to allow for attachment between the tube engaging element and the patient attachment element, including mechanically interlocking features, magnetic elements, adhesives, and the like. The patient attachment 320 may be various shapes, thicknesses, and densities. For example, the patient attachment element 320 may be oblong with a non-constant radii, as depicted in FIG. 6. In other examples, the patient attachment element 320 may be circular with a constant radius, square, rectangular, or any other shape that allows for attachment to a patient.

The patient attachment element 320 may also include a plurality of layers. Such layers may include a fabric or paper liner with adhesive backing 330. Such a lining 330 may be a removable peel-away liner that when removed exposes adhesive backing. The adhesive layer may be on either side of the patient attachment element 320, which may allow for the tube stabilization patch to be adhered to the interior or exterior of the medical garment (discussed below) or directly adhered to the patient. Such layers may also include an absorption layer 340, which may collect, absorb, or otherwise contain residues, bodily fluids, excess adhesive, or other containments that it may come into contact with. By absorbing unwanted contaminants, the absorption layer 340 further reduces the risk of infection at the entry point of the catheter by reducing the amount of non-sterile contact the entry point may come into contact with. Such layers may also include an antibacterial or antimicrobial layer 350, which may include conventional antibacterial or antimicrobial substances to further reduce the risk of infection and/or presence of bacterial or microbial contaminants.

The patient attachment element 320 may include an adhesive, magnetic, or structural elements to provide a detachable connection with the medical garment 100. In such embodiments, the medical garment 100 may include complementary surfaces that may allow for an adhesive, magnetic, or structural element to adhere to the medical garment. This may assist keeping the tube stabilization patch in a comfortable place on the patient.

The patient attachment element 320 may also include a pull tab 360 for easy removal of the tube stabilization patch 300, such that the patient may apply retracting pressure to the pull tab 360 to separate the adhesive from the patient, allowing for easy removal.

The tube stabilization patch 300 may include an access separation 370. The access separation 370 may allow for attachment or detachment of the tube stabilization patch from around the medical tubing or catheter. The access separation 370 may extend from the openable bore to an edge of the patient attachment element 320, such an extension may cut through a radial line of the patient attachment element from the tube engaging element 310, through an external perimeter of the patient attachment element 320. The access separation 370 may cut through all or a portion of the plurality of layers, allowing for a medical tube or catheter to be passed through the separation and secured by the tube engaging element, without removing the catheter.

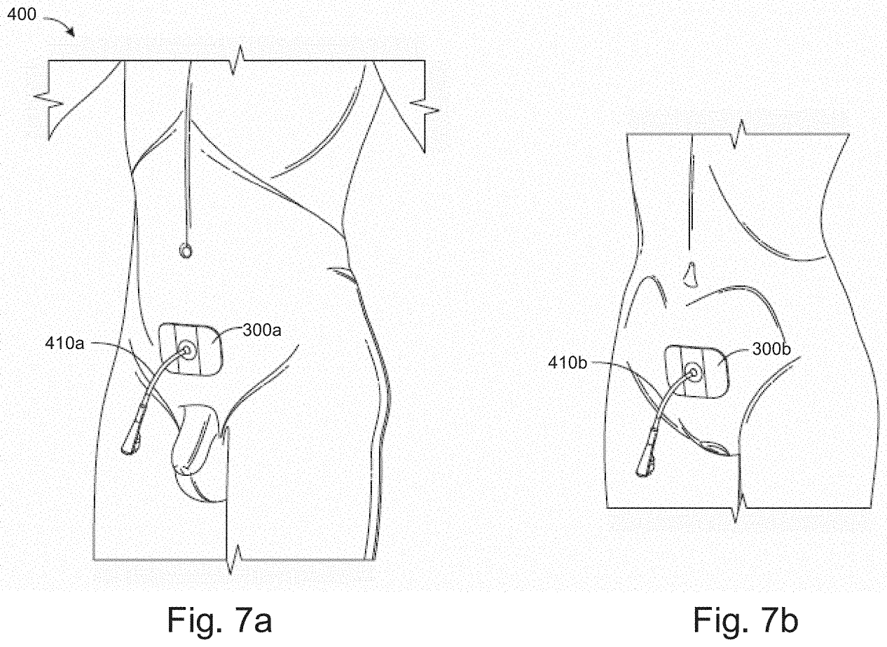

FIGS. 7a & 7b are example views of patients with suprapubic or ileostomy catheters secured by a tube stabilization patch of a device or system for stabilizing medical tubing protruding from a patient, according to embodiments of the present disclosure.

According to various embodiments of the present disclosure, the tube stabilization patch 300a, b may be attached to a catheter and secured on the body of the patient. As depicted in FIGS. 7a & 7b, the tube stabilization patch may be used with a variety of different types of medical tubing and/or catheters (i.e., suprapubic or ileostomy catheters 410a, b). The tube stabilization patch 300 may be separated at the access separation (370, FIG. 6) and slid over the medical tubing or catheter without removing the medical tubing or catheter or disconnected the medical tubing or catheter from any drainage bags. Once it is slid over the medical tubing or catheter, the tube stabilization patch 300 may be secured or adhered to the body of the patient by removing the adhesive backing (330, FIG. 6).

The tube stabilization patch 300 reduces the amount of relative movement when worn by preventing the medical tubing or catheter from moving in and out (pistoning) of the patient, and adding further rigidity to the lateral movement of the medical tubing or catheter due to the stiffer tube engaging element (310, FIG. 6). The tube engaging element may be more rigid that conventional medical tubing or catheters.

In various embodiments, once the medical tubing, for example, an in-dwelling catheter, is positioned in the patient, the tube engagement element may be attached to the tube, for example by snapping onto, clamping it over, or securing it around, the medical tubing at a location at or near where the tubing exits the patient's body. The patient attachment element, which may be peripherally secured to the circumference of the tube engaging element, may then be secured, via adhesive or similar, to the skin of the patient. In other words, once the medical tubing is positioned and secured in the tube engaging element of the tube stabilization patch, the tube stabilization patch may be secured to the body of the patient.

The application of the tube stabilization patch 300 to suprapubic or ileostomy catheters in FIGS. 7a & 7b is merely exemplary. One skilled in the art would understand how such a tube stabilization patch 300 may be applied to other medical tubing and/or catheters.

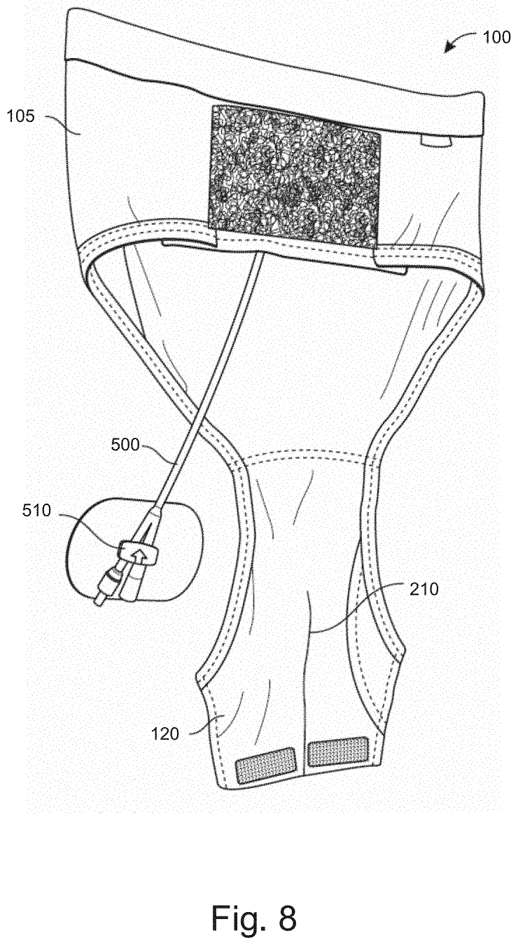

FIG. 8 is a front view of an example medical garment of a device or system for stabilizing medical tubing protruding from a patient with an adjustable flap in an unattached position and a catheter securing mechanism in a secured position with a closure flap closed, with example medical tubing protruding from a patient, according to embodiments of the present disclosure.

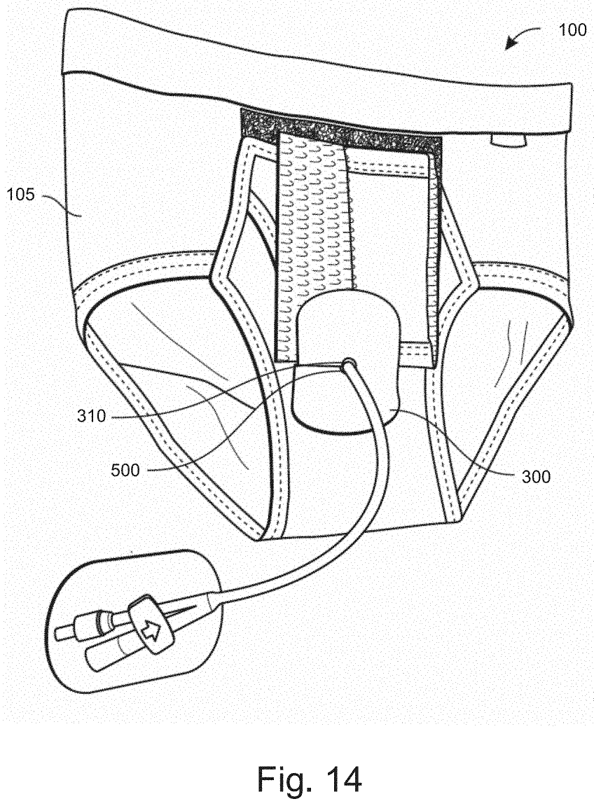

According to various embodiments of the present disclosure, the medical garment 100 may be donned with medical tubing or a catheter 500 already in place. For example, a conventional Foley catheter (e.g., medical tubing or a catheter 500) may have a Foley catheter securement device 510 that is secured to one of the patient's legs. In various embodiments, the present disclosure may work in conjunction with such a Foley catheter system. In such an embodiment, the Foley catheter may be inserted by a medical professional, then the tube stabilization patch may be attached to the patient (as discussed), and the medical garment 100 may be donned. Additionally, the Foley catheter may be placed, the medical garment may be donned, and the tube stabilization patch may then be adhered to the exterior of the medical garment and secured around the catheter, as further discussed in relation to FIG. 14 below.

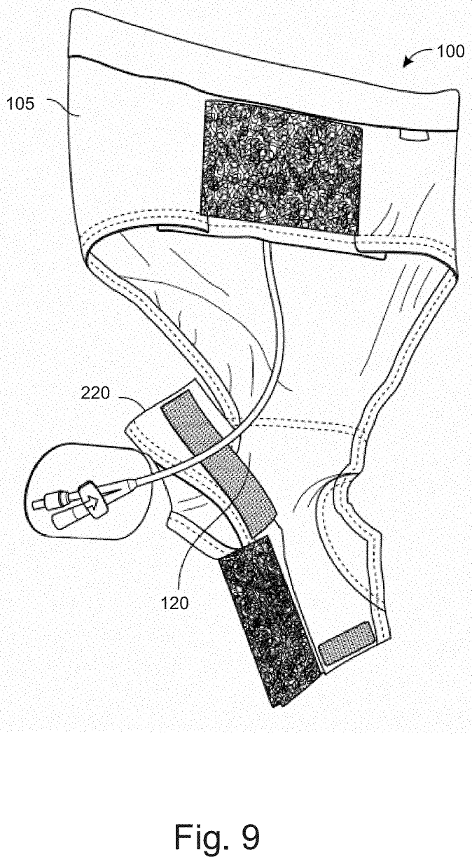

FIG. 8 depicts an initial step of donning the medical tubing stabilization device. Such that, when the device is initially worn, the patient may don the medical garment 100, verify that the body of the medical garment 105 is secured around the lower abdomen of the patient, locate the adjustable flap 120 and locate and position the catheter exit port 210. The patient may then place the medical tubing or catheter through the securing mechanism as discussed in relation to FIG. 9.

FIG. 9 is a perspective view of an example medical garment of a device or system for stabilizing medical tubing protruding from a patient with an adjustable flap in an unattached position and a catheter securing mechanism in an unsecured position with a closure flap open, wherein example medical tubing protruding from a patient is passed between a first portion and a second portion of the catheter securing mechanism, according to embodiments of the present disclosure.

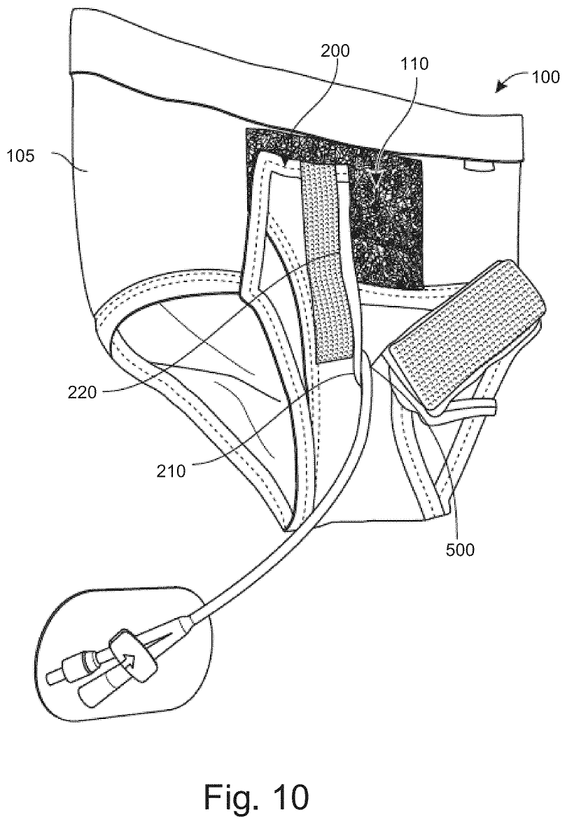

According to various embodiments of the present discloser, the portion of the adjustable flap may be slide between the catheter (secured to the patient's leg on one side and inside the patient on the other) and the body of the patient. Moreover, the medical tubing or catheter may be disposed through the opening in the securing mechanism on the adjustable flap 120. Such disposition may occur by moving the medical tubing or catheter in between the first securing portion 220 and the second securing portion 230 or by moving the first securing portion 220 between the medical tubing or catheter and the body of the patient. The medical tubing or catheter may be secured or rest on or in the catheter exit port as described in relation to FIG. 10.

Referring to FIG. 10, this figure depicts a perspective view of an example medical garment of a device or system for stabilizing medical tubing protruding from a patient with an adjustable flap in a partially attached position and a catheter securing mechanism in an unsecured position with a closure flap open, wherein example medical tubing protruding from a patient is passed between a first portion and a second portion of the catheter securing mechanism and situated at a catheter exit port, according to embodiments of the present disclosure.

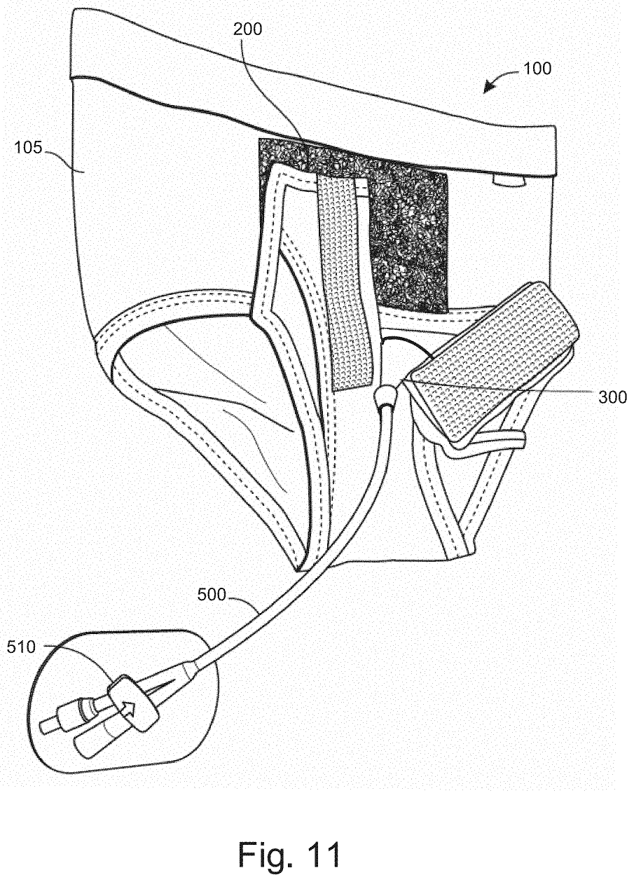

According to embodiments of the present disclosure, the medical tubing or catheter 500 may be disposed between the first securing portion 220 and the second securing portion 230 reaching the catheter exit port 210. The medical tubing or catheter 500 may be disposed on or at the catheter exit port 210 with the tube stabilization patch (as described in relation to FIG. 11) or without the tube stabilization patch, as depicted in FIG. 10. The system or device depicted in FIG. 10 is merely exemplary of the path a medical tubing or catheter may take to be secured in the medical garment 100, it does not, however, limit the disclosure, which is only limited by the claims.

Referring to FIG. 11, this figure depicts a perspective view of an example medical garment of a device or system for stabilizing medical tubing protruding from a patient with an adjustable flap in a partially attached position and a catheter securing mechanism in an unsecured position with a closure flap open, wherein example medical tubing protruding from a patient is passed through the tube stabilization patch and disposed between the body of the patient and the first and second portion of the catheter securing mechanism, according to embodiments of the present disclosure.

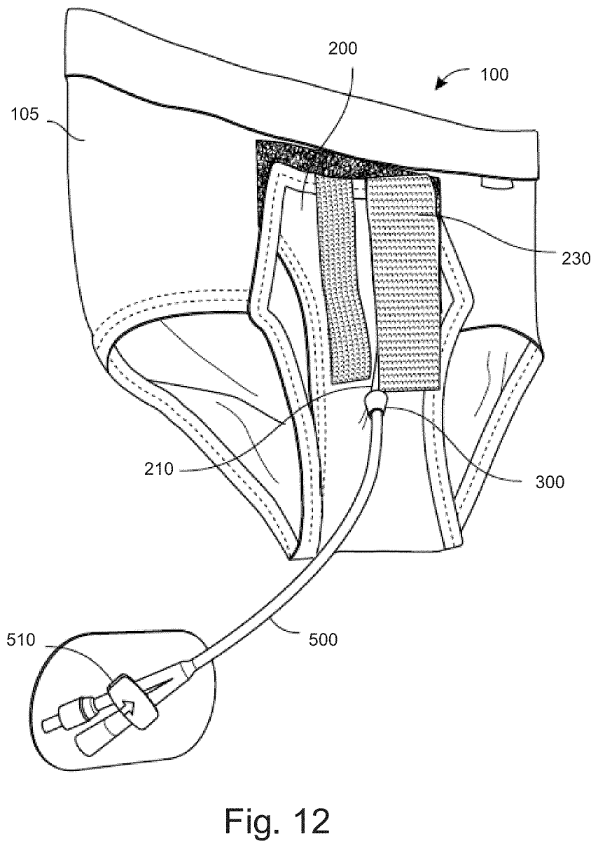

According to various embodiments of the present disclosure, the medical garment 100 may have the tube stabilization patch 300 placed behind the securing mechanism 200. When the tube stabilization patch 300 is placed behind the securing mechanism 200 and the securing mechanism is fully secured, the tube engagement element may protrude through the catheter exit port, such that the only portion of the tube stabilization patch that can be seen from the front of the medical garment is the tube engaging element. After the tube stabilization patch 300 has been placed in the catheter exit port, the adjustment flap 120 may be fully attached to the body 105 of the medical garment 100, as discussed in relation to FIG. 12.

Referring to FIG. 12, this figure depicts a perspective view of an example medical garment of a device or system for stabilizing medical tubing protruding from a patient with an adjustable flap in an attached position and a catheter securing mechanism in a secured position with a closure flap open, wherein example medical tubing protruding from a patient is passed through the tube stabilization patch and disposed between the body of the patient and the first and second portion of the catheter securing mechanism, according to embodiments of the present disclosure.

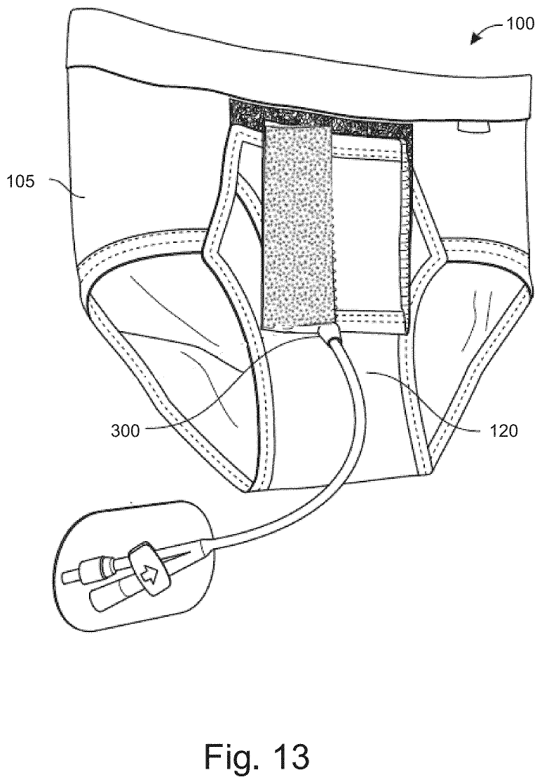

According to various embodiments of the present disclosure, the second securing portion 230 may be attached to the first attachment portion 110 once the tube stabilization patch 300 has been placed in the catheter exit port 210. Where the securing mechanism 200 is located or positioned may be determined or adjusted by both where the second securing portion 230 is placed on the first attachment portion 110 and by where the second securing portion 230 is placed on the first securing portion 220, as discussed in relation to FIG. 13.

Referring to FIG. 13, this figure depicts a perspective view of an example medical garment of a device or system for stabilizing medical tubing protruding from a patient with an adjustable flap in an attached position and a catheter securing mechanism in a secured position with a closure flap closed, wherein example medical tubing protruding from a patient is passed through the tube stabilization patch and disposed between the body of the patient and the first and second portion of the catheter securing mechanism, according to embodiments of the present disclosure.

According to various embodiments of the present disclosure, the medical garment 100 may be fully donned with the tube stabilization patch 300 in place, as depicted in FIG. 13. The second securing portion 230 may be moved from an unsecured position to a secured position by moving it across the front body 105 of the medical garment 100, to secure the second securing portion 230 to the first securing portion 220. By doing this, the catheter exit port 210 is still open at the base of the first and second securing portion 230, allowing the medical tubing or catheter to exit the medical garment and attach to the securement device on the patient's leg.

Referring to FIG. 14, this figure depicts a perspective view of an example medical garment of a device or system for stabilizing medical tubing protruding from a patient with an adjustable flap in an attached position and a catheter securing mechanism in a secured position with a closure flap closed, wherein example medical tubing protruding from a patient is passed through the first and second portion of the catheter securing mechanism and then passed through the tube stabilization patch with the tube stabilization patch adhered to an external portion of the medical garment, according to embodiments of the present disclosure.

According to various embodiments of the present disclosure, the medical garment 100 may be partially or fully donned prior to the placement of the tube stabilization patch 300. In such an embodiment, the body 105 of the medical garment 100 may be worn, and the adjustable flap may be secured with the catheter 500 disposed in the securing mechanism. The tube stabilization patch 300 may be adhered to an external surface of the adjustable flap, where the tube engaging element 310 can be secured around the catheter 500 protruding from both the patient and the medical garment 100.

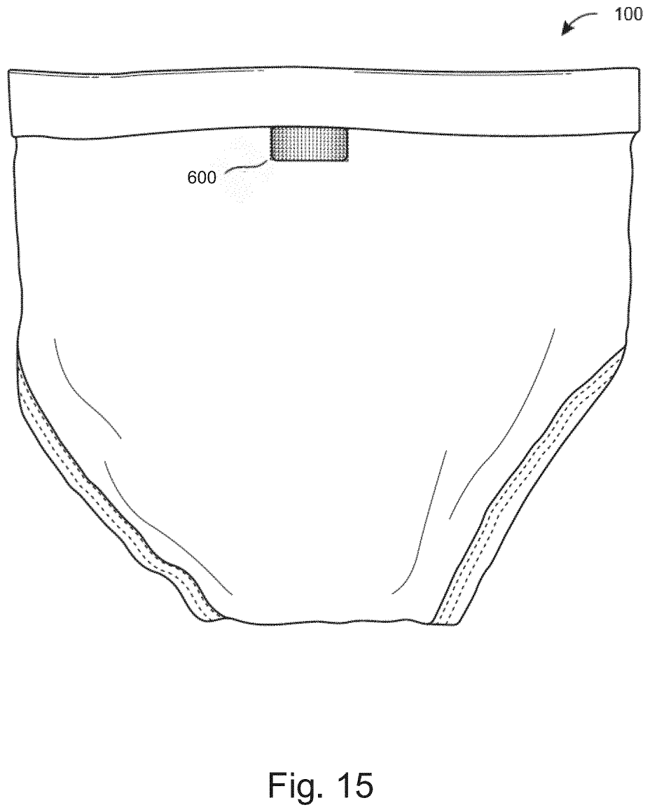

Referring to FIG. 15, this figure depicts a rear view of an example medical garment of a device or system for stabilizing medical tubing protruding from a patient with a posterior attachment member, according to embodiments of the present disclosure.

According to various embodiments of the present disclosure, the medical garment 100 may include a posterior attachment member 600. The posterior attachment member 600 may be a section of the posterior or rear portion of the medical garment 100. For example, the posterior attachment member 600 may be centrally located on the rear portion of the medical garment 100 or located in the middle-upper region of the medical garment (e.g., disposed approximately below the encircling waistband and approximately in the lateral center). The posterior attachment member 600 may have a width and height, which may be the same or different. For example, the width may be 2 inches and the height may be 1 inch. The posterior attachment member 600 may be a conventional hook and loop method, Velcro, reusable adhesive, and/or any other suitable method for repeatably attaching fabrics to one another.

The posterior attachment member 600 can hold or attach the adjustable flap to the rear portion of the medical garment 100 to allow the patient wearing the medical garment 100 to use the restroom, or otherwise access under the medical garment 100 without having to remove the body the medical garment, remove the medical tubing or catheter, or disconnect the catheter from a drainage bag.

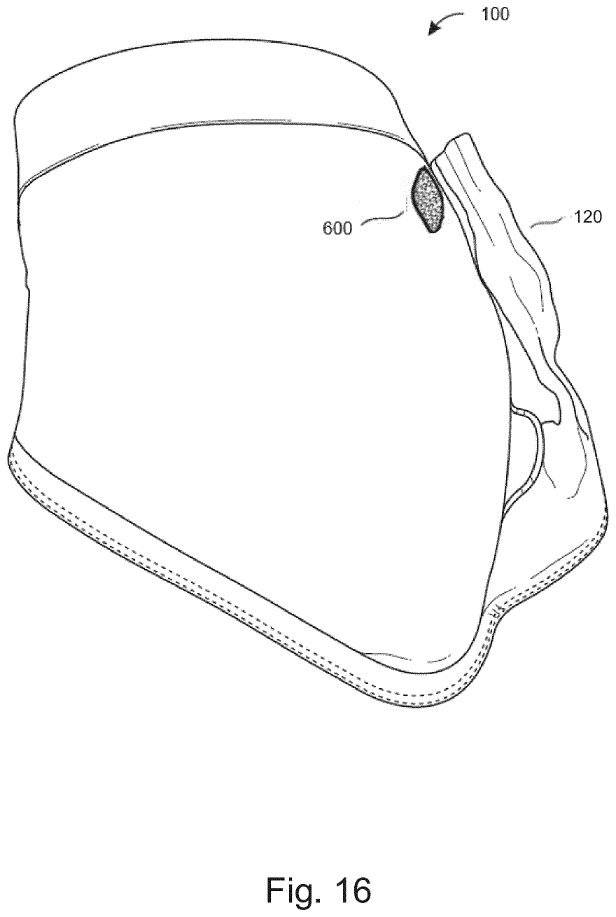

Referring to FIG. 16, this figure depicts a side view of an example medical garment of a device or system for stabilizing medical tubing protruding from a patient with an adjustable flap attached to the posterior attachment member, according to embodiments of the present disclosure.

According to various embodiments of the present disclosure, the medical garment 100 may have the adjustable flap 120 pulled between the legs of the patient, from where it attaches on the body (not shown) to the rear portion of the body where it can be attached to the posterior attachment member 600.

The medical tubing stabilization device may decrease the level of relative movement of medical tubing and catheters by stabilizing lateral, internal, and piston movement through the application of a tube stabilization patch and the further securing of the tube stabilization patch with the medical garment, as described in relation to various embodiments of the present disclosure.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical, or physical partitioning and configurations can be implemented to implement the desired features of the present disclosure. Also, a multitude of different constituent module names other than those depicted herein can be applied to the various partitions. Additionally, with regard to diagrams, operational descriptions, and method claims, the order in which the steps are presented herein shall not mandate that various embodiments be implemented to perform the recited functionality in the same order unless the context dictates otherwise.

Although the disclosure is described above in terms of various embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “typical,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “element” does not imply that the components or functionality described or claimed as part of the element are all configured in a common package. Indeed, any or all of the various components of an element can be combined in a single package or separately maintained and can further be distributed in multiple groupings or packages or across multiple locations.

Additionally, the various embodiments set forth herein are described in terms of exemplary diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A medical tubing stabilization device, comprising:
a tube stabilization patch comprising:
a tube engaging element configured to receive medical tubing;
a patient attachment element coupled to the tube engaging element, wherein the patient attachment element surrounds the tube engaging element; and
an access separation, wherein the access separation spans from an edge of the patient attachment element to the tube engaging element, wherein the medical tubing is passable through the access separation for the tube engaging element to receive the medical tubing;
a medical garment comprising:
a body that wraps around a patient; and
an adjustable flap connected to a first portion of the body, wherein the adjustable flap comprises:
an attachment mechanism that releasably connects the adjustable flap to a second portion of the body;
a securing mechanism comprising a first securing portion and a second securing portion, wherein the first securing portion is connectable to a second securing portion, wherein the securing mechanism is on a top portion of the adjustable flap; and
an exit port configured to receive the medical tubing or tube engaging element, wherein the exit port is between the first securing portion and the second securing portion.

2. The medical tubing stabilization device of claim 1, wherein the tube engaging element is configured to receive medical tubing through the access separation.

3. The medical tubing stabilization device of claim 1, wherein the attachment mechanism further comprises:
a first attachment portion, wherein the first attachment portion is connected to the second portion of the body of the medical garment; and
a second attachment portion, wherein the second attachment portion is connected to a back of the securing mechanism.

4. The medical tubing stabilization device of claim 1, wherein the securing mechanism further comprises:
a first securing portion, wherein the first securing portion makes up a first side of the securing mechanism; and
a second securing portion, wherein the second securing portion makes up a second side of the securing mechanism.

5. The medical tubing stabilization device of claim 1, wherein the exit port is disposed between the first securing portion and second securing portion.

6. The medical tubing stabilization device of claim 1, wherein the patient attachment element further comprises a plurality of layers.

7. A method of stabilizing medical tubing protruding from a patient with a medical tubing stabilization device, comprising:
applying a tube stabilization patch to medical tubing protruding from the patient, wherein applying the tube stabilization patch comprises:
passing medical tubing through an access separation to a tube engaging element, wherein the access separation spans from an edge of the tube stabilization patch to the tube engaging element;
securing medical tubing with the tube engaging element, wherein the tube engaging element is configured to receive medical tubing; and
applying a patient attachment element to the patient, wherein the patient attachment element is configured to couple to the periphery of the tube engaging element and be separated by the access separation;
donning a medical garment, wherein donning the medical garment, comprises:
wrapping a body portion around the patient;
adjusting an adjustable flap to attach to the body portion via an attachment mechanism, secure around the medical tubing via a securing mechanism, and receive the tube engaging element at an exit port, wherein the exit port is surrounded by the securing mechanism.

8. The method of stabilizing medical tubing protruding from a patient with a medical tubing stabilization device of claim 7, wherein securing medical tubing with the tube engaging element further comprises passing the medical tubing through the securing mechanism to reach the exit port.

9. The method of stabilizing medical tubing protruding from a patient with a medical tubing stabilization device of claim 7, wherein the access separation and tube engaging element is closed once the medical tubing is secured in the tube engaging element.

10. The method of stabilizing medical tubing protruding from a patient with a medical tubing stabilization device of claim 7, wherein applying the tube stabilization patch and donning the medical garment dampen movement on the body from the medical tubing.

11. The method of stabilizing medical tubing protruding from a patient with a medical tubing stabilization device of claim 7, wherein applying the tube stabilization patch further comprises removing a lining to expose an adhesive coating that adheres the tube stabilization patch to the patient.

12. The method of stabilizing medical tubing protruding from a patient with a medical tubing stabilization device of claim 7, wherein applying the tube stabilization patch further comprises removing a lining to expose an adhesive coating that adheres the tube stabilization patch to an external surface of the medical garment.

13. The method of stabilizing medical tubing protruding from a patient with a medical tubing stabilization device of claim 7, wherein applying the tube stabilization patch and donning the medical garment does not require removing the medical tubing from the patient or disconnecting the medical tubing from any drainage or collection bags.

14. A urinary catheter stabilization device, comprising:
a catheter stabilization patch comprising:
a catheter engaging element configured to receive a urinary catheter;
a patient attachment element coupled to the catheter engaging element, wherein the patient attachment element surrounds the catheter engaging element; and
an access separation, wherein the access separation spans from an edge of the patient attachment element to the catheter engaging element, wherein the urinary catheter is passable through the access separation for the catheter engaging element to receive the medical tubing;
a medical undergarment comprising:
a body that wraps around a patient; and
an adjustable flap connected to a first portion of the body, wherein the adjustable flap comprises:
an attachment mechanism that releasably connects the adjustable flap to a second portion of the body, wherein the adjustable flap has a first width and a first height and the second portion has a second width and a second height, these widths and heights allow for the adjustable flap to be attaches in various positions allowing for a neutral position of the catheter in patients of various anatomies;
a securing mechanism comprising a first securing portion and a second securing portion, wherein the first securing portion is connectable to a second securing portion, wherein the securing mechanism is on a top portion of the adjustable flap; and
an exit port configured to receive the catheter or the catheter engaging element, wherein the exit port is between the first securing portion and the second securing portion.

15. The urinary catheter stabilization device of claim 14, wherein the catheter engaging element is configured to receive in-dwelling urinary catheters, suprapubic catheters, or ileostomy catheters through the access separation.

16. The urinary catheter stabilization device of claim 14, wherein the attachment mechanism further comprises:
a first attachment portion, wherein the first attachment portion is connected to the second portion of the body of the medical garment; and
a second attachment portion, wherein the second attachment portion is connected to a back of the securing mechanism.

17. The urinary catheter stabilization device of claim 14, wherein the securing mechanism further comprises:
a first securing portion, wherein the first securing portion makes up a first side of the securing mechanism; and
a second securing portion, wherein the second securing portion makes up a second side of the securing mechanism.

18. The urinary catheter stabilization device of claim 14, wherein the exit port is disposed between the first securing portion and second securing portion.

19. The urinary catheter stabilization device of claim 14, wherein the patient attachment element further comprises a plurality of layers, such layers including an adhesive layer, a removable liner, an absorption layer, and an antimicrobial layer.

20. The urinary catheter stabilization device of claim 14, wherein the catheter engaging element is comprised of a resilient, single-use material.

21. The urinary catheter stabilization device of claim 14, wherein the medical undergarment further comprises an encircling waistband and an encircling body.

22. A medical tubing stabilization device, comprising:
a tube stabilization patch comprising:
a tube engaging element configured to receive medical tubing;
a patient attachment element coupled to the tube engaging element, wherein the patient attachment element surrounds the tube engaging element; and
an access separation, wherein the access separation spans from an edge of the patient attachment element to the tube engaging element, wherein the medical tubing is passable through the access separation for the tube engaging element to receive the medical tubing.

23. The medical tubing stabilization device of claim 22, wherein the tube engaging element is configured to receive medical tubing through the access separation.

24. The medical tubing stabilization device of claim 22, wherein the exit port is disposed between the first securing portion and second securing portion.

25. The medical tubing stabilization device of claim 22, wherein the patient attachment element further comprises a plurality of layers.
